(19) United States
(12) Patent Application Publication
(10) Pub. No.: US 2007/0036842 A1
(43) Pub. Date: Feb. 15, 2007

(54) Non-woven scaffold for tissue engineering

(75) Inventor: Randal W. Spencer, Providence, RI (US)

Correspondence Address: EDWARDS & ANGELL, LLP, P.O. BOX 55874, BOSTON, MA, 02205, US

(73) Assignee: Concordia Manufacturing LLC, Coventry, RI (US)

(21) Appl. No.: 11/204,655

(22) Filed: Aug. 15, 2005

(51) Int. Cl. A61K 35/12; A01N 1/02; A61K 9/70

(52) U.S. Cl. 424/443; 424/93.7; 435/2

(57) ABSTRACT

Scaffolds for tissue engineering, and methods and systems for manufacturing the same. The starting material used to manufacture the scaffolds may include a needled non-woven sheet, or a batt of fibers. In either case, the starting material includes a bioabsorbable polymer, such as polyglycolic acid or poly-L-lactide. This allows the scaffold to dissolve when exposed to water (e.g., when placed in the body). The starting material is needled into a three dimensional shape that is substantially seamless and then set with heat and pressure to provide substantially uniform thickness and density. This affords an improved substrate on which to place cells that will form the tissue.

### TECHNICAL FIELD

The present invention relates generally to tissue engineering and, more specifically, to three dimensional scaffolds used to support cell growth.

### BACKGROUND INFORMATION

Tissue engineering is the development and manipulation of laboratory-grown cells to replace or support defective or injured body parts. It involves the reproduction of functional tissue outside the body using properly selected cells as seed material. Once the tissue matures to an appropriate level, it is implanted into the body where, ideally, it matures further and becomes integrated with the body. Tissues that form some or all of the heart, bladder, vertebrae, and other vital organs are candidates for tissue engineering. See, for example, G. Matsumura, Successful Application of Tissue Engineered Vascular Autografts: Clinical Experience, 24 Biomaterials 2303 (2003). Further, tissue engineering to support bone reconstruction is of interest, because bone, which ordinarily heals when fractured, is not regenerated in an adult human when lost by disease.

Generally, the seed cells must be placed on some form of structure that will support them and shape them into the tissue of interest. These supports, known as “tissue engineering scaffolds,” were developed from absorbable polymers utilizing textile processing expertise to create non-woven substrates. For organs having simple shapes, a single-piece substrate can be adequate to guide the cells into the proper form. Nevertheless, organs having complex shapes can require multi-piece substrates that are attached together to create a scaffold to emulate the required profile. Even some single-piece substrates may need to be urged into a specific profile and, therefore, may require some form of attachment between different parts of the substrate. In either case, attaching the substrates can be accomplished by conventional means, such as stitching (i.e., suturing), but this can have a detrimental effect on the tissue generation process. For example, the areas of the attachments (i.e., “seams”) present discontinuities of the surface of the scaffold and can exhibit a density different than that of other, undisturbed parts of the scaffold. This may retard or inhibit the growth of cells near or on the seams.

From the foregoing, it is apparent that there is a need for tissue engineering scaffolds having complex shapes to support the growth of vital organs. The scaffolds should be substantially uniform to ensure proper growth of the seed cells.

### SUMMARY OF THE INVENTION

The present invention relates to tissue engineering scaffolds that can have complex shapes, and the systems and methods for manufacturing them. The resulting scaffolds are substantially seamless and do not present discontinuities that can impede proper growth of the seed cells.

The invention features a method where starting material used to manufacture the scaffold is manipulated into a three dimensional shape by having one part of the material overlap with another part of the material and “needled.” Needling is a process of joining materials whereby one or more barbed needles are repeatedly pressed into and removed from the materials. In certain embodiments, the overlapping areas are tapered so, when they are needled, their overall thickness is about the same as non-needled areas of the material.

The starting material can be a needled non-woven sheet or, in some embodiments, can be a batt of fibers. In further embodiments, the starting material includes a bioabsorbable polymer, such as polyglycolic acid, or poly-L-lactide, or copolymers that include one of each.

The invention also features scaffolds for receiving the living cells, the scaffolds having needled areas and, in other embodiments, non-needled areas. The scaffolds can be made from one or more pieces of starting material, and can include a bioabsorbable polymer.

Another embodiment of the invention includes a system for manufacturing the scaffolds. The system features a flexible mandrel that can exhibit a profile that corresponds to some, if not all, of the profile of the scaffold. The flexible mandrel can include one or more support ribs that are driven by actuators to urge the mandrel into a particular shape (e.g., the desired shape of the scaffold). The support ribs, which can be similar to leaf springs, include at least one perforated rib. Needling is accomplished by driving one or more barbed needles through the starting material that is placed between the barbed needles and the perforated rib while the starting material is moved relative to the needles.

Following needling, the resulting scaffold should be “set” with heat and pressure to control the thickness and density of the finished product. Accordingly, an embodiment of the invention includes a method of setting complex and three dimensional scaffolds using a heated mold and an expandable bladder.

Given the sophistication of the shapes of the scaffolds and the need for precise control over their manufacture, embodiments of the invention include a controller, typically a personal computer, that directs the operation of the manufacturing apparatus, provides operational feedback to an operator, and allows the operator to create and modify the manufacturing process as required.

Other aspects and advantages of the present invention will become apparent from the following detailed description, taken in conjunction with the accompanying drawings, illustrating the principles of the invention by way of example only.

### BRIEF DESCRIPTION OF THE DRAWINGS

The foregoing and other objects, features, and advantages of the present invention, as well as the invention itself, will be more fully understood from the following description of various embodiments, when read together with the accompanying drawings, in which:

FIG. 1 is a flowchart depicting a method for making a shape for receiving one or more living cells in accordance with an embodiment of the invention;

FIG. 2 is a flowchart depicting a method for making a shape for receiving one or more living cells in accordance with another embodiment of the invention;

FIGS. 3A, 3B, and 3C are schematic views depicting a structure for receiving one or more living cells in accordance with an embodiment of the invention;

FIGS. 4A, 4B, 4C, and 4D are schematic views depicting a structure for receiving one or more living cells in accordance with another embodiment of the invention;

FIGS. 5A, 5B, 5C, and 5D are schematic views depicting a system for manufacturing a shape for receiving one or more living cells in accordance with an embodiment of the invention;

FIGS. 6A and 6B are schematic views depicting a system for manufacturing a shape for receiving one or more living cells in accordance with another embodiment of the invention;

FIG. 7 is a schematic view depicting a system for manufacturing a shape for receiving one or more living cells in accordance with a further embodiment of the invention;

FIG. 8 is a schematic view depicting a system for manufacturing a shape for receiving one or more living cells in accordance with an additional embodiment of the invention;

FIG. 9 is a schematic view depicting an atrial branch created using a structure for receiving one or more living cells manufactured in accordance with another embodiment of the invention; and

FIG. 10 is a schematic view depicting an intervertebral disk created using a structure for receiving one or more living cells manufactured in accordance with another embodiment of the invention.

### DESCRIPTION

As shown in the drawings for the purposes of illustration, the invention can be embodied in methods and systems for creating a three dimensional shape using bioabsorbable material that serves as a scaffold for tissue generation. Embodiments of the invention are useful because they allow the rapid growth of cells to form an organized three dimensional tissue structure. The porous nature of the shapes manufactured according to the invention further facilitates the cell growth. (The shapes typically have a porosity factor greater than about 97%.)

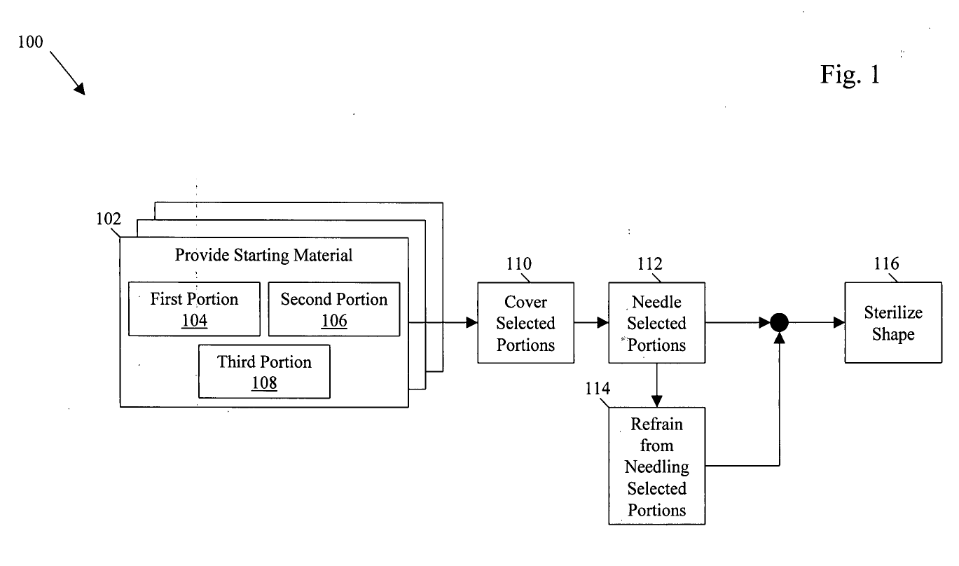

In brief overview, FIG. 1 is a flowchart depicting a method 100 for making at least a portion of a shape for receiving one or more living cells in accordance with an embodiment of the invention. The method includes a step of first providing one or more pieces of a starting material (STEP 102; depicting several pieces of starting material) that will serve as the substrate for the cell growth. In some embodiments, the starting material can be a needled non-woven sheet, sometimes referred to as “felt.” In other embodiments, the starting material can be a batt of fibers, sometimes known as “carded fibers.” Methods of producing the starting material are like those employed in the textile arts; that is, using carding and needle punching procedures. These procedures are known and will not be described in detail herein. Nevertheless, in some embodiments, it is desirable to control the alignment of the fibers in the batt. These fibers can be substantially aligned with one another or randomly oriented with respect to one another. Substantial alignment can be achieved by, for example, passing the batt of fibers through a carding machine. This will substantially align the fibers in what is typically called the “machine direction” (i.e., the direction of travel through the machine). Random orientation can be achieved by, for example, passing the batt of fibers through the carding machine a second time but in a direction that is different from that of the first pass (e.g., by rotating the batt ninety degrees relative to its orientation during the first pass before passing it through the carding machine the second time).

The starting material, whether it includes felt or a batt of fibers, in some embodiments includes a bioabsorbable polymer. The bioabsorbable polymer reacts with water and bodily fluids and dissolves. The bioabsorbable polymer can begin to dissolve before and/or after being placed in the body. This may be controlled by selecting bioabsorbable polymers that have different absorption rates, depending on the nature of the tissue being reconstructed. This is desirable because the scaffold for tissue generation created according to the invention should, when implanted, disappear over time, leaving the three dimensional tissue structure in the body. Typical bioabsorbable polymers include, for example, polyglycolic acid, or poly-L-lactide, or copolymers that include one of each. In some embodiments, long fiber reinforcements are added inside the non-woven structure. By aligning these long fibers in appropriate directions, the strength of the scaffold can be increased without reducing its porosity.

Next, the starting material is manipulated (STEP 110) such that a first portion 104 of the starting material covers a second portion 106 of the starting material. This leaves a third portion 108 of the starting material uncovered by either the first portion 104 or the second portion 106. Also, the third portion 108 does not cover the first portion 104 or the second portion 106. A result of this overlapping is the generation of a three dimensional shape that includes an interior space (e.g., a cavity). (In embodiments where two or more pieces of starting material are used, the pieces are manipulated until a portion of one piece covers a portion of a second piece, leaving one or more portions on one or more of the pieces uncovered. Also, the uncovered portion does not cover any portion of the pieces.)

Next, the now overlapping first portion 104 and the second portion 106 are needled (STEP 112). Needling is a process of joining materials whereby one or more barbed needles are repeatedly pressed into and removed from the materials. The needles catch small amounts of the materials, and their penetration into and removal from the materials compact (i.e., consolidate) the materials and increase entanglement within the materials, thereby joining them. In some embodiments, the needling (STEP 112) is performed over an area that is larger or smaller than that defined by the overlapping first portion 104 and the second portion 106. Indeed, all of the starting material can be needled. Nevertheless, in other embodiments, portions of the starting material are prevented from being needled (STEP 114). For example, at least some of the third portion 108, which does not overlap, and is not overlapped by, other portions of the starting material, is not needled.

The needling (STEP 112) results in the joining of the overlapping first portion 104 and the second portion 106. An advantage of needling is that it joins these portions without creating a seam. Conventional methods of joining the portions (e.g., stitching) generally create a seam, which can be problematic when present in a scaffold for tissue generation. For example, sutures used to sew the seam do not necessarily absorb at the same rate as the remainder of the scaffold, since they typically have a larger dimension than the fibers making up the scaffold. Further, the act of stitching causes the density of the scaffold to change in the stitched region, thereby making it more difficult for growing cells to penetrate the scaffold. A typical outcome of the needling (STEP 112) is that the once separate portions blend together to create a virtually homogeneous structure. The resulting three dimensional shape formed according to an embodiment of the invention has substantially no seams and a generally consistent density.

After forming, the shape is generally sterilized (STEP 116) before it receives the living cells. In various embodiments, the sterilization (STEP 116) can be accomplished by exposing the shape to one or more of ethylene oxide (sometimes referred to as “ETO”), gamma radiation, or electron beam irradiation.

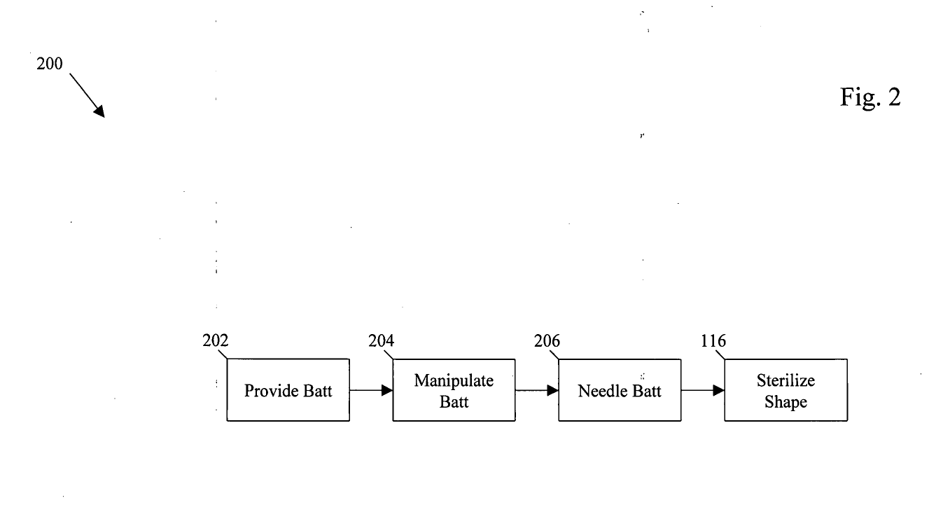

FIG. 2 is a flowchart depicting a method 200 for making at least a portion of a shape for receiving one or more living cells in accordance with another embodiment of the invention. In this embodiment, a batt of fibers is provided (STEP 202) as starting material. As described above, the fibers can be substantially aligned with one another or randomly oriented with respect to one another. Next, the batt is manipulated into a three dimensional structure that includes an interior space (e.g., a cavity) (STEP 204), and then the entirety of the batt is needled (STEP 206). The resulting three dimensional structure is substantially seamless and homogeneous, and is generally sterilized (STEP 116) before use.

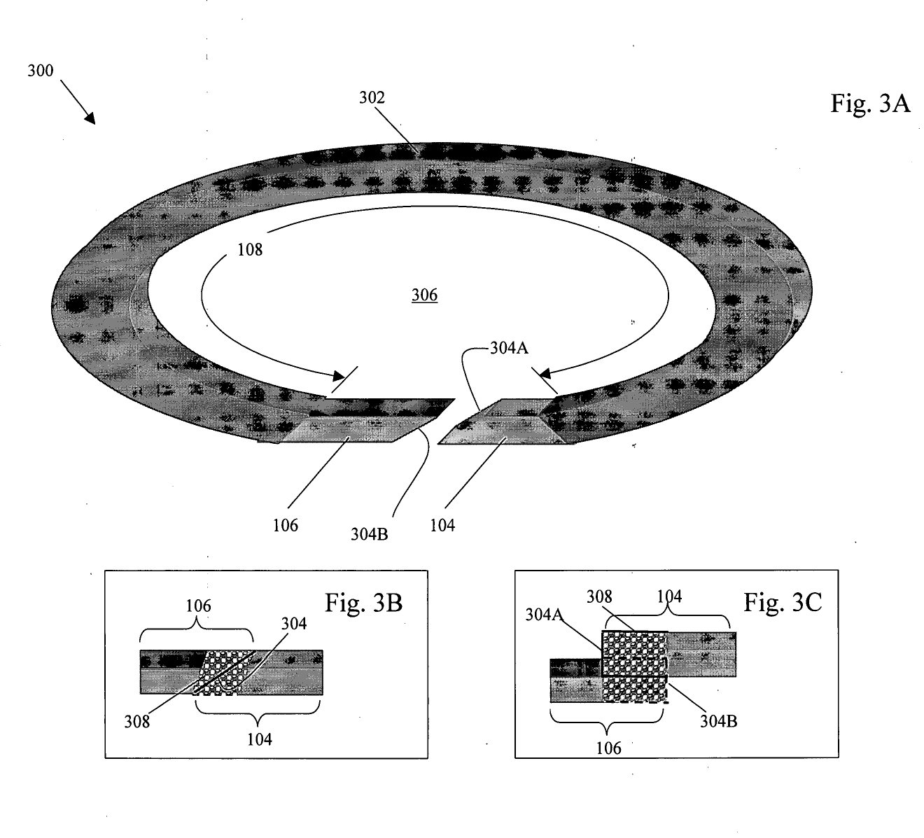

In brief overview, FIGS. 3A, 3B, and 3C are schematic sectional views depicting a structure 300 for receiving one or more living cells in accordance with an embodiment of the invention. The structure 300 includes one or more pieces of starting material 302 formed into a three dimensional configuration that includes an interior space 306 (e.g., a cavity). As described above, the starting material can be a needled non-woven sheet (e.g., “felt”) or a batt of fibers (e.g., “carded fibers”), and can include a bioabsorbable polymer, such as polyglycolic acid, or poly-L-lactide, or copolymers that include one of each.

The starting material 302 includes a first portion 104, a second portion 106, and a third portion 108. The structure 300 is configured such that the first portion 104 and the second portion 106 overlap. Further, neither the first portion 104 nor the second portion 106 cover (i.e., overlap) the third portion 108. Also, the third portion 108 does not cover either the first portion 104 or the second portion 106.

The first portion 104 has an edge 304A. The second portion 106 has an edge 304B. In some embodiments, the edges 304A, 304B (collectively, 304) are tapered. FIG. 3B shows a typical linear tapered edge configuration. Of course, other edge shapes (i.e., tapers), such as those including a contour (e.g., an arcuate section with a particular radius of curvature) are possible. When the tapered edges 304 are overlapped, the taper allows the combined thickness of the first portion 104 and second portion 106 to be substantially equal to a thickness of the third portion 108. In other words, the area of the structure 300 where portions overlap is not substantially thicker than an area of the structure 300 where there is no overlap. Alternatively, FIG. 3C depicts an embodiment where the edges 304 are not tapered. In this case, the area of the structure 300 where portions overlap is thicker than an area of the structure 300 where there is no overlap.

The structure 300 also includes a needled region 308 that is subjected to the needling as described above. The needled region 308 includes the first portion 104 and the second portion 106. Joining the first portion 104 and the second portion 106 by needling results in structure 300 that is substantially seamless. Note that the structure 300 includes a non-needled region that includes at least a part of the third portion 108.

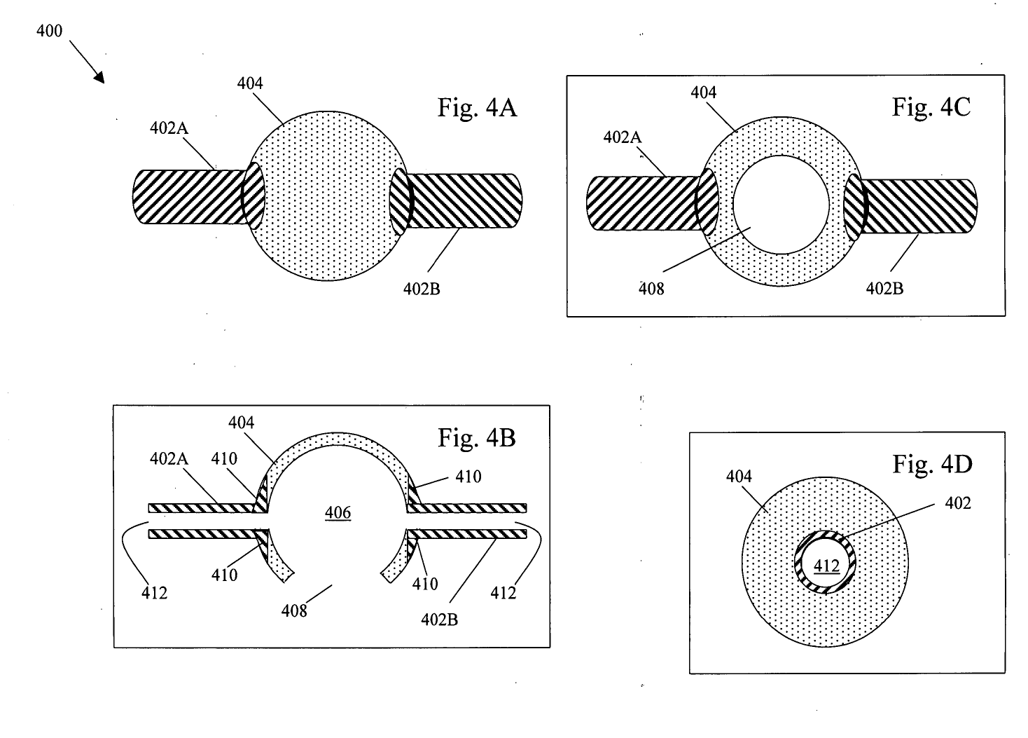

FIGS. 4A, 4B, 4C, and 4D are schematic views depicting another structure 400 for receiving one or more living cells in accordance with another embodiment of the invention. Specifically, FIG. 4A represents a top view, FIG. 4B represents a side cross sectional view, FIG. 4C represents a bottom view, and FIG. 4D represents a side view. These figures show two tubular structures 402A, 402B (collectively, 402) attached to a chamber 404 having an interior space 406 (i.e., a cavity) and an opening 408. The chamber 404 can be any three dimensional shape but, for simplicity, FIGS. 4A, 4B, 4C, and 4D depict a spherical shape.

As shown in FIG. 4B, the tubular structures 402 have an interior space 412 (e.g., a lumen) that is in communication with the interior space 406 of the chamber 404. Consequently, fluids, gases, etc., are able to pass through the and into and out of the chamber 404. Such a structure could be used, for example, as the scaffold to engineer tissue for chambers of the heart (with blood vessels entering and leaving the heart), stomach, intestines , and a bladder (with ureters entering, or the urethra leaving the bladder, or both).

In some embodiments, the tubular structures 402 are attached to the chamber 404 using the needling process described above. In further embodiments, where the tubular structures 402 and the chamber 404 are attached in various locations, the edges 410 of each are tapered. Consequently, at each location of attachment, the combined thickness of the structure 400 is substantially equal to the thickness of the tubular structures 402 or the chamber 404 alone.

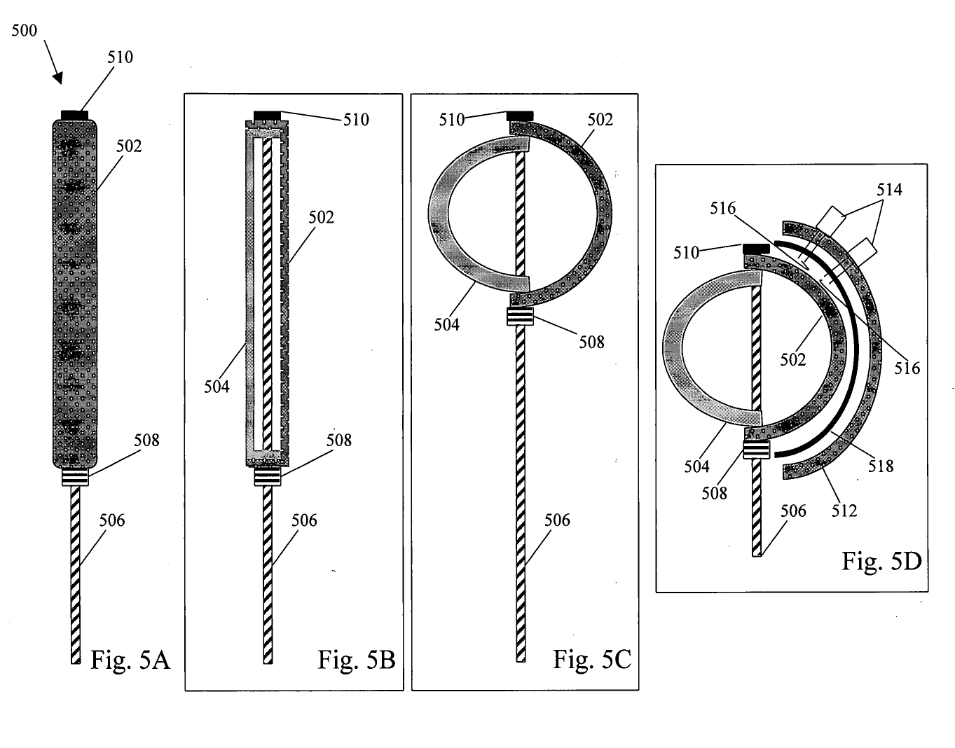

In brief overview, FIG. 5A depicts a flexible mandrel 500 used in a system to manufacture a shape for receiving one or more living cells in accordance with an embodiment of the invention. FIG. 5B represents a side cross sectional view of the flexible mandrel 500 in a relaxed state. FIG. 5C represents a side cross sectional view of the flexible mandrel 500 in a tensioned state. The flexible mandrel 500 includes a first support rib 502 that is typically perforated and a second support rib 504 that may or may not be perforated. (FIG. 5B shows a non-perforated version of the second support rib 504.) The first and second support ribs 502, 504 are typically manufactured from a flexible and resilient material, such as, for example, stainless steel, carbon, or a composite. Manufacturing of the shape typically occurs in a cleanroom. This allows control of the environment to, for example, keep particulates and moisture at their appropriate levels.

The first and second support ribs 502, 504 are mounted axially on a support rod 506, which can be threaded. An adjustment control 508 is also mounted on the support rod 506 proximate to one end of the support ribs 502, 504. The adjustment control 508 can be moved along at least part of the length of the support rod 506. (If the support rod 506 is threaded, the adjustment control 508 can be a nut that mates with and traverses the thread.)

A fixed end cap 510 is mounted on the support rod 506 proximate to the other end of the support ribs 502, 504. In this configuration, given the flexibility of the support ribs 502, 504, movement of the adjustment control 508 along the support rod 506 toward the fixed end cap 510 causes the support ribs 502, 504 to bow in an outward direction, as shown in FIG. 5C. Movement of the adjustment control 508 along the support rod 506 away from the fixed end cap 510 causes the support ribs 502, 504 to return to a relaxed state. Accordingly, the support rod 506 and the adjustment control 508 collectively operate as an actuator to impart a profile on the flexible mandrel 500.

In some embodiments, as shown in FIG. 5D, an outer form 512 is disposed about the outwardly bowed support rib 502. The outer form 512 is typically perforated and bears one or more needling assemblies 514. Each needling assembly 514 typically includes one or more barbed needles 516 and means for moving the needles 516 in a back and forth motion through the perforations in the outer form 512, such as a reciprocating motor (not shown). The outer form 512 is oriented such that the needles 516 pass through the perforations of the outwardly bowed support rib 502. Consequently, starting material 518 (e.g., a non-woven sheet or batt of fibers) for making the shape for receiving the cells can be placed on the flexible mandrel 500, after which the support ribs 502, 504 are bowed in an outward direction by the actuator, and then needled as shown in FIG. 5D. During needling, the needles 516 pass back and forth through (i) the perforations in the outer form 512, (ii) the starting material 518, and (iii) the perforations in the support rib 502. To needle all of the required areas, the starting material 518 is moved relative to the needling assembly 514. This can be accomplished by moving the starting material 518 while keeping the support rib 502 and the outer form 512 stationary. It can also be accomplished by moving the support rib 502 and the outer form 512 and keeping the starting material 518 stationary. (To allow for proper travel of the needles 516, the support rib 502 and the outer form 512 should remain stationary relative to each other, thereby keeping the perforations in each aligned.) Means for moving the support rib 502, the outer form 512, and the starting material 518 include a servomotor, or stepper motor, or both, as well as a powered set of rollers, one with a “north-south” axis and the other with an “east-west axis”. The rollers contact the starting material 518 and their controlled rotation appropriately positions the starting material 518. Once the needling is complete, the actuator relaxes the support ribs 502, 504, thereby allowing removal of the starting material 518.

A controller can direct the overall operation of the flexible mandrel 500, support rod 506 and the adjustment control 508 (i.e., the actuator), needling assembly 514, needles 516, and hardware for moving the starting material 518, support rib 502 and the outer form 512. A controller can be a personal computer configured with typical memory, mass storage, input-output, and communication-networking (e.g., Internet) devices. The controller, under software control, can coordinate the operation of these elements and report their status to an operator. The operator can receive this information locally or over a distance by virtue of the communication-networking devices. The operator can direct the operation of the controller (e.g., program it) locally or over a distance as well.

FIGS. 5A, 5B, 5C, and 5D depict a flexible mandrel 500 that includes only two support ribs 502, 504. Other configurations that include a different number of support ribs are within the scope of the invention. In particular, multiple support ribs may be arranged to exhibit a particular profile when tensioned. The profile can correspond to the desired profile of some or all of the shape that will receive the living cells. The desired profile can be symmetrical, non-symmetrical, axi-symmetrical, or non-axi-symmetrical.

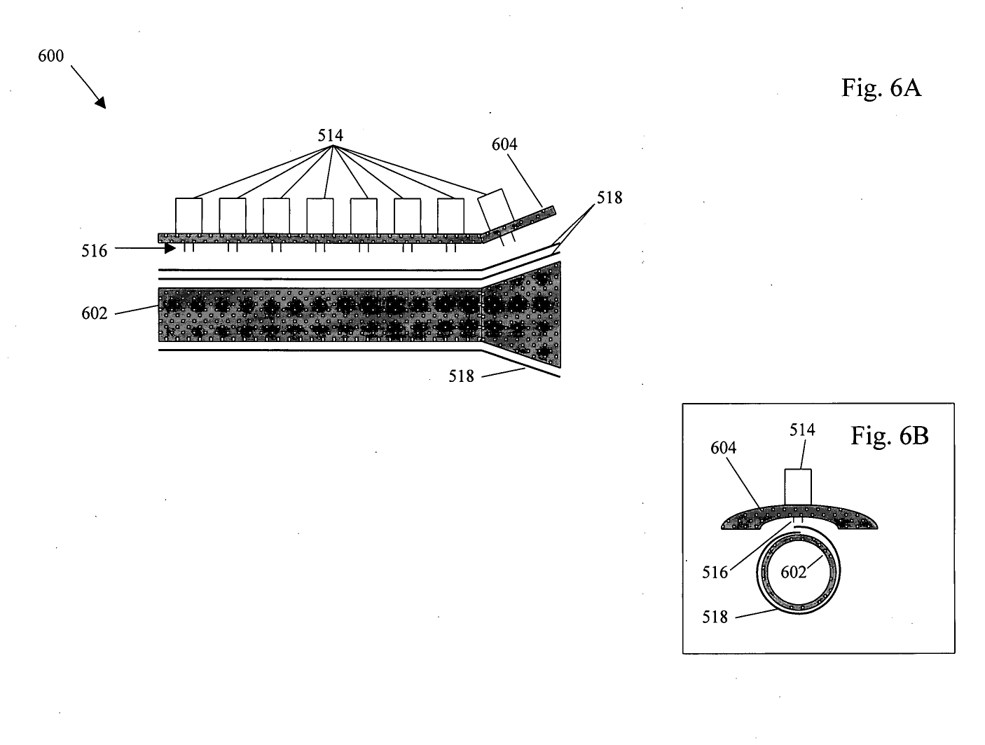

In some embodiments, cylindrical shapes, like the tubular structures 402, can be manufactured using the apparatus 600 depicted in FIG. 6A. The apparatus 600 includes an inner form 602 that is perforated. An outer form 604 is disposed adjacent to the inner form 602. The outer form 604 is typically perforated and bears one or more needling assemblies 514. Each needling assembly 514 typically includes one or more barbed needles 516 and means for moving the needles 516 in a back and forth motion through the perforations in the outer form 512, such as a reciprocating motor (not shown). The outer form 512 is oriented such that the needles 516 pass through the perforations of the inner form 602.

Starting material 518 (e.g., a non-woven sheet or batt of fibers) for making the shape for receiving the cells can be placed on the inner form 602 such that the ends of the starting material 518 overlap and needled as shown in FIG. 6B. During needling, the needles 516 pass back and forth through (i) the perforations in the outer form 604, (ii) the starting material 518, and (iii) the perforations in the inner form 602. To needle all of the required areas, the starting material 518 is moved relative to the needling assembly 514. This can be accomplished by moving the starting material 518 while keeping the inner form 602 and the outer form 604 stationary. It can also be accomplished by moving the inner form 602 and the outer form 604 and keeping the starting material 518 stationary. (To allow for proper travel of the needles 516, the inner form 602 and the outer form 604 should remain stationary relative to each other, thereby keeping the perforations in each aligned.) Means for moving the inner form 602, the outer form 604, and the starting material 518 include a servomotor, or stepper motor, or both, as well as a powered set of rollers as described above. Once the needling is complete, the starting material 518, now needled into a cylindrical shape, is removed from the inner form 602.

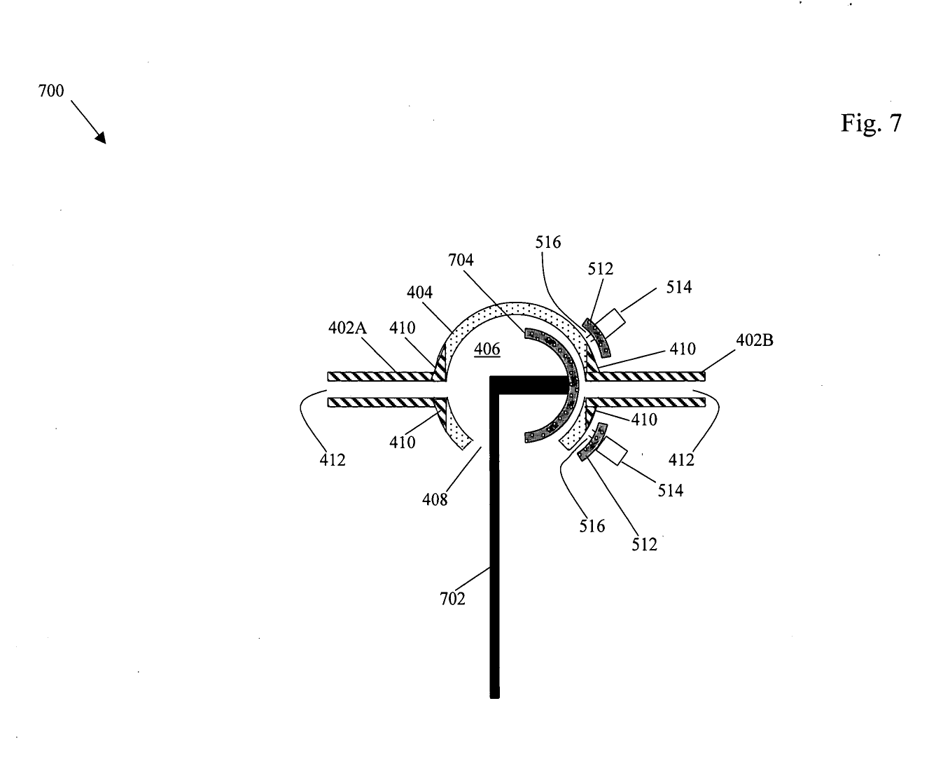

A cylindrical shape can be attached to a spherical shape using the apparatus 700 depicted in FIG. 7. The resulting arrangement can be similar to that of structure 400, as shown in FIGS. 4A, 4B, 4C, and 4D. These figures show two tubular structures 402A, 402B (collectively, 402) attached to a chamber 404 having an interior space 406 (i.e., a cavity) and an opening 408. The apparatus 700 includes a form support 702 that is attached to an inner form 704 that is perforated. The chamber 404 is placed on the inner form 704 by way of the opening 408 such that the area where the chamber 404 is to attach to the tubular structures 402 is substantially adjacent to the inner form 704. One of the tubular structures 402B is also proximate this area. As described above, the edges 410 of the chamber 404 and the tubular structures 402 can be tapered.

Similar to the embodiments described above, an outer form 512 that is typically perforated is disposed about the inner form 704 such that the perforations of each are aligned. Consequently, during needling, the needles 516 pass back and forth through the perforations and the area where the chamber 404 is to attach to the tubular structure 402B.

Once needling has been completed and the shape for receiving the cells is complete, a final setting process typically occurs. Setting allows precise control over the thickness and density of the needled material. The setting process generally includes pressing the material with a load of twenty to fifty tons at a temperature that is between the glass transition and melting temperatures of the material (e.g., from about 38 degrees C. (100 degrees F.) to about 260 degrees C. (500 degrees F.)). The setting process usually takes five to ten minutes, and is typically performed in two minute increments. The final thickness is generally ranges from about one to about five millimeters. The final density generally ranges from about 32 to about 400 milligrams per cubic centimeter.

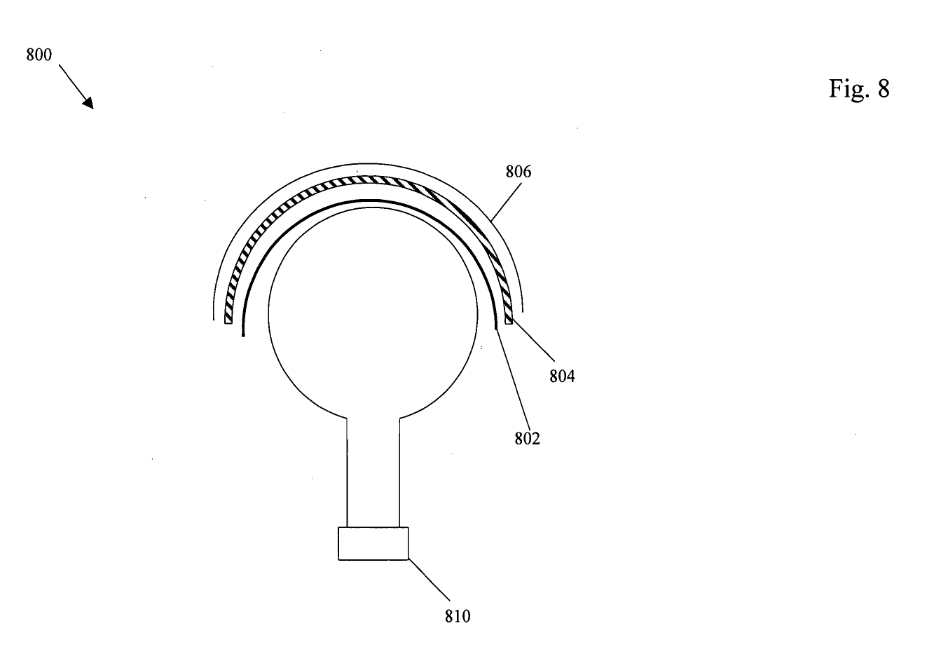

FIG. 8 depicts an apparatus 800 for setting needled material 808 that does not have a planar profile (e.g., that includes cylindrical or spherical shapes). The apparatus 800 includes an expandable bladder 802 that is connected to a supply 810 of gas (e.g., air) or liquid at a controlled pressure. Disposed about the bladder 802 is a mold 804 that, in some embodiments, is manufactured from stainless steel. Adjacent to the mold 804 is a heating element 806 that heats the mold 804 to a particular temperature (e.g., a temperature that is between the glass transition and melting temperatures of the needled material 808).

During use, the needled material 808 is placed on the bladder 802 while the latter is deflated. The heating element 806 warms the mold 804 to the desired temperature (e.g., about 65 degrees C. (150 degrees F.)), and the gas or liquid flows into the bladder 802, pressurizing and expanding it using a pressure of about 2700 kPa (400 pounds psi) to about 3400 kPa (500 pounds psi). This presses the needled material 808 into the heated mold 804. After a sufficient amount of time (e.g., five to ten minutes), the bladder 802 is deflated and the needled material 808 is removed from the apparatus 800. This process sets the needled material 808, thereby providing the desired control over its thickness and density.

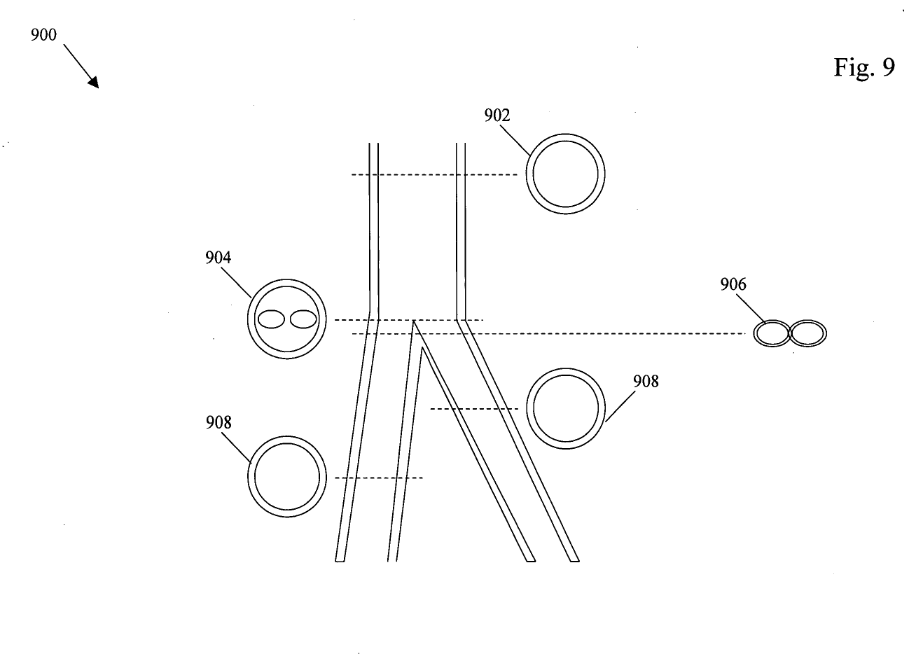

As described above, scaffolds manufactured according to embodiments of the invention may be used to engineer tissue for different organs. For example, FIG. 9 is a schematic depiction of an atrial branch 900 showing the divergence of one blood vessel into two blood vessels. The single blood vessel has a cross section 902 at the location of the corresponding dashed line in FIG. 9. At the beginning of the divergence, the structure has cross section 904, which shows the initial formation of the two divergent blood vessels. The vessels further diverge, resulting in cross section 906, and ultimately form two independent blood vessels, each having cross section 908.

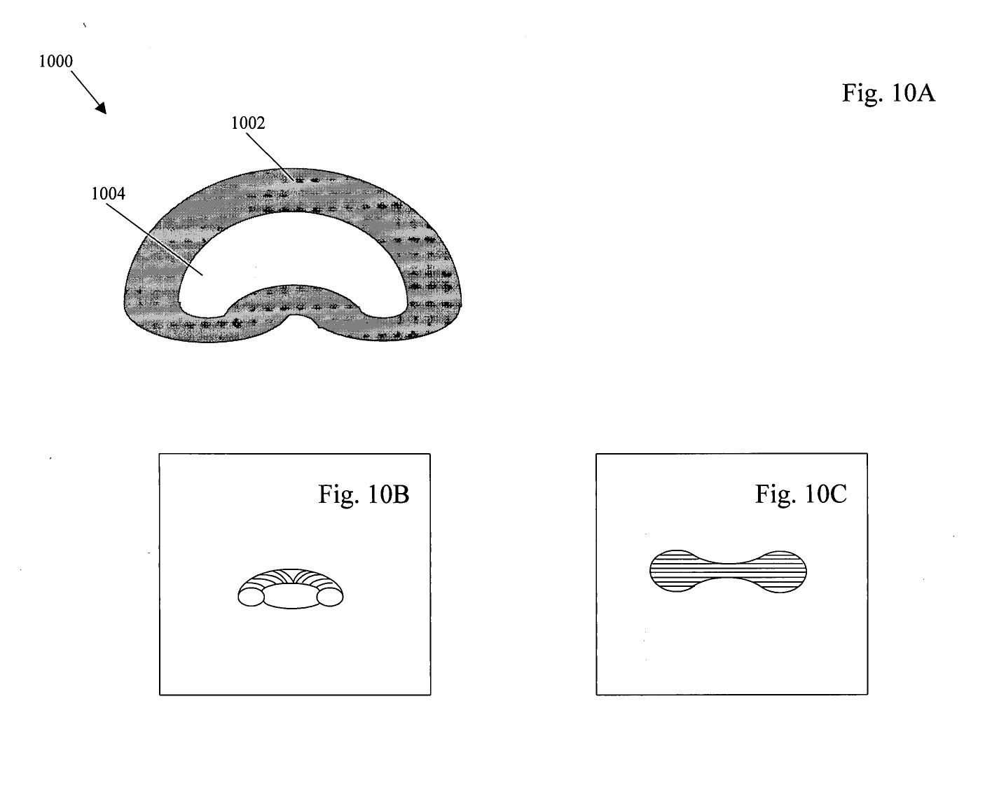

Another example of an organ is the intervertebral disk 1000 depicted in schematic form in FIG. 10A. The disk 1000 includes an annulus fibrosus portion 1002, which is a fibrocartilageous ring that surrounds the gelatinous nucleus pulposus portion 1004. Cross sections of the disk 1000 are shown in schematic form in FIGS. 10B and 10C, which also show the tapering and layering nature of the disk 1000. Tissue engineering scaffolds manufactured according to an embodiment of the invention can emulate this and, in particular, can be manufactured such that the needling extends to only selected layers, which can be fewer than the total number of layers present.

From the foregoing, it will be appreciated that systems and methods according to the invention afford a simple and effective way to generate scaffolds for tissue engineering that can have complex shapes, and yet are substantially uniform to promote desired cell growth.

The invention can be embodied in other specific forms without departing from the spirit or essential characteristics thereof. The foregoing embodiments are to be considered in all respects illustrative rather than limiting of the invention.

What is claimed is:

1. A method of making at least a portion of a shape for receiving one or more living cells, the method comprising the steps of:
providing a piece of starting material including a first portion, a second portion, and a third portion;
covering the first portion with the second portion, neither the first portion nor the second portion covering the third portion, the third portion not covering either the first portion or the second portion;
needling at least the first and second portions to attach at least the first and second portions to each other to create a three dimensional shape that defines an interior space; and
refraining from needling at least a portion of the third portion.

2. The method of claim 1 wherein the first and second portions each include a taper.

3. The method of claim 2 wherein a combined thickness of the first and second portions is substantially equal to a thickness of the third portion.

4. The method of claim 1 wherein the providing step comprises providing a needled non-woven sheet as the piece of starting material.

5. The method of claim 1 wherein the providing step comprises providing a batt of fibers as the piece of starting material.

6. The method of claim 1 wherein the needling step results in the creation of the three dimensional shape with substantially no seams.

7. The method of claim 1 wherein the piece of starting material comprises a bioabsorbable polymer.

8. The method of claim 7 wherein the polymer comprises at least one of polyglycolic acid or poly-L-lactide.

9. The method of claim 1 further comprising the step of sterilizing the shape.

10. A method of making at least a portion of a shape for receiving one or more living cells, the method comprising the steps of:
providing a plurality of pieces of starting material, each of the pieces including a first portion and a second portion;
covering the first portion of a first one of the pieces with the first portion of a second one of the pieces;
needling at least the first portions of the first and second ones of the pieces to attach at least the first portions to each other to create at least a portion of a three dimensional shape that defines an interior space; and
refraining from needling at least a portion of the second portion of at least one of the plurality of pieces.

11. The method of claim 10 wherein each of the first portions includes a taper.

12. The method of claim 11 wherein a combined thickness of the first portions is substantially equal to a thickness of the second portion.

13. The method of claim 10 wherein the providing step comprises providing a first needled non-woven sheet as the first one of the pieces and providing a second needled non-woven sheet as the second one of the pieces.

14. The method of claim 10 wherein the providing step comprises providing a first batt of fibers as the first one of the pieces and providing a second batt of fibers as the second one of the pieces.

15. The method of claim 10 wherein the needling step results in the creation of the three dimensional shape with substantially no seams.

16. The method of claim 10 wherein the covering step comprises covering the first portion of the first one of the pieces with the first portion of the second one of the pieces such that the second portion of at least one of the first and second ones of the pieces (i) is not covered and (ii) does not cover the first portion of either the first or second ones of the pieces.

17. The method of claim 10 wherein at least one of the plurality of pieces of starting material comprises a bioabsorbable polymer.

18. The method of claim 17 wherein the polymer comprises at least one of polyglycolic acid or poly-L-lactide.

19. The method of claim 10 further comprising the step of sterilizing the shape.

20. A method of making at least a portion of a shape for receiving one or more living cells, the method comprising the steps of:
providing a plurality of pieces of starting material, each of the pieces including a first portion and a second portion, wherein each of the first portions includes a taper;
covering the first portion of a first one of the pieces with the first portion of a second one of the pieces, wherein a combined thickness of the first portions is substantially equal to a thickness of the second portion; and
needling at least the first portions of the first and second ones of the pieces to attach at least the first portions to each other to create at least a portion of a three dimensional shape that defines an interior space.

21. The method of claim 20 wherein the providing step comprises providing a first needled non-woven sheet as the first one of the pieces and providing a second needled non-woven sheet as the second one of the pieces.

22. The method of claim 20 wherein the providing step comprises providing a first batt of fibers as the first one of the pieces and providing a second batt of fibers as the second one of the pieces.

23. The method of claim 20 wherein the needling step results in the creation of the three dimensional shape with substantially no seams.

24. The method of claim 20 wherein the covering step comprises covering the first portion of the first one of the pieces with the first portion of the second one of the pieces such that the second portion of at least one of the first and second ones of the pieces (i) is not covered and (ii) does not cover the first portion of either the first or second ones of the pieces.

25. The method of claim 20 wherein at least one of the plurality of pieces of starting material comprises a bioabsorbable polymer.

26. The method of claim 25 wherein the polymer comprises at least one of polyglycolic acid or poly-L-lactide.

27. The method of claim 20 further comprising the step of sterilizing the shape.

28. A method of making at least a portion of a shape for receiving one or more living cells, the method comprising the steps of:
providing at least one batt of fibers;
manipulating the at least one batt into a three dimensional structure, the three dimensional structure defining an interior space; and
needling the at least one batt to consolidate the fibers and create the three dimensional structure.

29. The method of claim 28 wherein the needling step results in the creation of the three dimensional structure with substantially no seams.

30. The method of claim 28 wherein the at least one batt comprises a bioabsorbable polymer.

31. The method of claim 30 wherein the polymer comprises at least one of polyglycolic acid or poly-L-lactide.

32. The method of claim 28 further comprising the step of sterilizing the shape.

33. A structure for receiving one or more living cells, comprising:
a piece of starting material formed into a three dimensional structure defining an interior space, the piece of starting material including a first portion, a second portion, and a third portion, the second portion covering the first portion, neither the first portion nor the second portion covering the third portion, the third portion not covering either the first portion or the second portion;
a needled region including the first portion and the second portion and with substantially no seams; and
a non-needled region including at least a portion of the third portion.

34. The structure of claim 33 wherein the first and second portions each include a taper.

35. The structure of claim 34 wherein a combined thickness of the first and second portions is substantially equal to a thickness of the third portion.

36. The structure of claim 33 wherein the piece of starting material comprises a needled non-woven sheet.

37. The structure of claim 33 wherein the piece of starting material comprises a batt of fibers.

38. The structure of claim 33 wherein the piece of starting material comprises a bioabsorbable polymer.

39. The structure of claim 38 wherein the polymer comprises at least one of polyglycolic acid or poly-L-lactide.

40. A structure for receiving one or more living cells, comprising:
a plurality of pieces of starting material formed into a three dimensional structure defining an interior space, each of the pieces including a first portion and a second portion, the first portion of a first one of the pieces covering the first portion of a second one of the pieces;
a needled region including the first portions of the first and second ones of the pieces and with substantially no seams; and
a non-needled region including at least a portion of the second portion of at least one of the plurality of pieces.

41. The structure of claim 40 wherein each of the first portions includes a taper.

42. The structure of claim 41 wherein a combined thickness of the first portions is substantially equal to a thickness of the second portion.

43. The structure claim 40 wherein at least one of the plurality of pieces of starting material comprises a needled non-woven sheet.

44. The structure of claim 40 wherein at least one of the plurality of pieces of starting material comprises a batt of fibers.

45. The structure of claim 40 wherein at least one of the plurality of pieces of starting material comprises a bioabsorbable polymer.

46. The structure of claim 45, wherein the polymer comprises at least one of polyglycolic acid or poly-L-lactide.

47. A structure for receiving one or more living cells, comprising:
a plurality of pieces of starting material formed into a three dimensional structure defining an interior space, each of the pieces including a first portion having a taper and a second portion, the first portion of a first one of the pieces covering the first portion of a second one of the pieces, wherein a combined thickness of the first portions is substantially equal to a thickness of the second portion; and
a needled region including the first portions of the first and second ones of the pieces and with substantially no seams.

48. The structure of claim 47 wherein at least one of the plurality of pieces of starting material comprises a needled non-woven sheet.

49. The structure of claim 47 wherein at least one of the plurality of pieces of starting material comprises a batt of fibers.

50. The structure of claim 47 wherein at least one of the plurality of pieces of starting material comprises a bioabsorbable polymer.

51. The structure of claim 50, wherein the polymer comprises at least one of polyglycolic acid or poly-L-lactide.

52. A system for manufacturing a shape for receiving one or more living cells, the system comprising:
a flexible mandrel including a plurality of support ribs for receiving at least one piece of starting material, wherein one of the support ribs defines a plurality of apertures;
a plurality of barbed needles disposed proximate to the support rib having the plurality of apertures;
at least one actuator associated with at least one of the support ribs;
means for pressing and removing the plurality of barbed needles through the at least one starting material and into the plurality of apertures; and
means for moving the at least one starting material relative to the support rib having the plurality of apertures.

53. The system of claim 52 wherein the at least one starting material comprises a non-woven sheet.

54. The system of claim 52 wherein the at least one starting material comprises a batt of fibers.

55. The system of claim 52 wherein the piece of starting material comprises a bioabsorbable polymer.

56. The system of claim 55 wherein the polymer comprises at least one of polyglycolic acid or poly-L-lactide.

57. The system of claim 52 wherein the flexible mandrel is characterized at least in part by a profile corresponding to at least a portion of the shape for receiving one or more living cells.

58. The system of claim 52 further comprising a controller in communication with the flexible mandrel, the plurality of barbed needles, the at least one actuator, the means for pressing and removing the plurality of barbed needles, and the means for moving the at least one starting material.

59. A method of setting at least a portion of a needled non-woven shape for receiving one or more living cells, the method comprising the steps of:
providing a mold;
heating the mold to a predetermined temperature;
providing an expandable bladder disposed within the mold and defining a gap therebetween;
placing the shape on the expandable bladder and within the gap; and
expanding the bladder to a predetermined pressure to cause the sheet to contact the mold for a predetermined period.
